(19) United States
(12) Patent Application Publication
(10) Pub. No.: US 2005/0038411 A1
(43) Pub. Date: Feb. 17, 2005

(54) Catheter

(76) Inventor: Yosuke Okada, Shizuoka-ken (JP)

Correspondence Address: LORUSSO, LOUD & KELLY, 3137 Mt. Vernon Avenue, Alexandria, VA, 22305, US

(21) Appl. No.: 10/900,087

(22) Filed: Jul. 28, 2004

(30) Foreign Application Priority Data
Aug. 12, 2003 (JP) 2003-292398

(51) Int. Cl. A61M 25/00

(52) U.S. Cl. 604/523; 600/433

(57) ABSTRACT

A catheter including a tubular body having a distal end, and a tubular tip portion extending contiguously from the distal end substantially coaxially therewith and terminating in a forward facing aperture, wherein the tubular tip portion is formed of a water-absorbing, water-swelling material.

### CROSS-REFERENCE TO RELATED APPLICATION

This application claims, under 35 USC 119, priorities of Japanese Patent Application No. 2003-292398, filed Aug. 12, 2003, disclosures of which, inclusive of the specification, claims and drawings, are hereby incorporated by reference herein.

### BACKGROUND OF THE INVENTION

1. Field of the Invention

This invention relates to a catheter for insertion into and placement in a blood vessel or other body cavity.

2. Description of Prior Art

Medical procedures using catheters are now widely adopted for various purposes such as for administration of a medicament solution or a nutrient solution, sampling of body fluids such as blood, and drainage and recycling of blood for dialysis. Such catheters have various structures including single lumen, double lumen and multi-lumen structures.

A catheter is generally inserted into a body cavity such as a blood vessel by a method using an introducer sheath. In this method, the introducer sheath is first inserted through the skin and underlying tissue into a blood vessel. The catheter is then inserted through the introducer sheath into the blood vessel. When it is necessary to insert the catheter into a specific distal site, a guide wire is inserted and is advanced to the site. The catheter is subsequently inserted along the guide wire. The above method is generally employed for intravascular arterial diagnosis and therapy.

A Seldinger method is also widely employed for the insertion of a catheter into a body cavity. A needle is used to pierce a path into the blood vessel. A guide wire is inserted through the needle and into the blood vessel after which the needle is withdrawn over the guide wire. The catheter is then passed over the guide wire. Finally, the guide wire is removed leaving the catheter in position within the vessel. In this case, in order for the catheter to be smoothly inserted into the blood vessel without injuring tissues, the tip portion of the catheter is narrowed so that the gap between the catheter and the guide wire is as small as possible. This method is widely used in venous catheter insertion.

When a catheter is placed in a blood vessel such that the tip end thereof is oriented at an angle with respect to the wall of the blood vessel, the wall is susceptible to be injured upon movement of the blood vessel caused as a result of throb. Also, when a catheter with its tip end having been placed in position is relocated and when the tip end thereof is oriented at an angle with respect to the wall of the blood vessel, the catheter tip is apt to penetrate through the blood vessel wall. In order to avoid such injury to the blood vessel walls, a thought may occur to round the tip of the catheter. With this method, however, it is impossible to prevent the occurrence of injury, since the catheter is thin.

To prevent injury to the blood vessel walls, catheters having a soft tip are proposed in U.S. Pat. No. 5,045,072 and U.S. Pat. No. 5,300,032. Because of lack of toughness, however, it is difficult to insert such a soft tip catheter through the skin and underlying tissue into blood vessel.

Japanese Unexamined Patent Publication No. H05-137791 discloses a catheter made from a polymeric composition which is sufficiently stiff in the dry state during insertion, but which softens and swells on hydration in the body. Because of low mechanical strengths of the polymeric composition, the catheter is usable only for a limited purpose.

### SUMMARY OF THE INVENTION

It is, therefore, an object of the present invention to provide a catheter which can be easily inserted into a blood vessel or other body cavity and which does not cause injury of blood vessel walls when the blood vessel moves by throb or when the catheter is displaced for relocation.

Another object of the present invention is to provide a catheter of the above-mentioned type which can provide sufficient fluid flow at a tip end portion thereof even when applied to a Seldinger method.

In accordance with one aspect of the present invention, there is provided a catheter comprising a tubular body having a distal end, and a tubular tip portion extending contiguously from said distal end substantially coaxially therewith and terminating in a forward facing aperture, said tubular tip portion being formed of a water-absorbing, water-swelling material.

In another aspect, the present invention provides a double lumen catheter, comprising a tubular body, and a planar septum disposed in said tubular body to divide the interior of said tubular body into a first lumen and a second lumen, said first and second lumens terminating in distally forward facing apertures such that the aperture of said second lumen is spaced in the distally forward of the aperture of said first lumen, wherein a tip portion of said tubular body which defines, together with said planar septum, a distal end part of said first lumen including said aperture thereof is formed of a composition comprising a water-absorbing, water-swelling resin.

### BRIEF DESCRIPTION OF THE DRAWINGS

Other objects, features and advantages of the present invention will become apparent from the detailed description of the preferred embodiments of the invention which follows, when considered in the light of the accompanying drawings, in which:

FIG. 1(a) is a partial cross-sectional view diagrammatically illustrating one embodiment of a catheter, before insertion, according to the present invention;

FIG. 1(b) is a partial cross-sectional view diagrammatically illustrating the catheter of FIG. 1(a) after absorption of water;

FIG. 2 is a partial cross-sectional view diagrammatically illustrating another embodiment of a catheter according to the present invention;

FIG. 3 is a partial cross-sectional view diagrammatically illustrating a further embodiment of a catheter according to the present invention;

FIG. 4 is a partial cross-sectional view diagrammatically illustrating a further embodiment of a catheter according to the present invention;

FIG. 5(a) is a partial cross-sectional view diagrammatically illustrating a fitting step (engaging step) in a method of preparing a catheter according to the present invention;

FIG. 5(b) is a partial cross-sectional view diagrammatically illustrating a catheter produced by a method shown in FIG. 5(a);

FIG. 6(a) is a partial cross-sectional view diagrammatically illustrating an abutting step (engaging step) in another method of preparing a catheter according to the present invention;

FIG. 6(b) is a partial cross-sectional view diagrammatically illustrating a catheter produced by a method shown in FIG. 6(a);

FIG. 7(a) is a partial cross-sectional view diagrammatically illustrating an abutting step (engaging step) in a further method of preparing a catheter according to the present invention;

FIG. 7(b) is a partial cross-sectional view diagrammatically illustrating a catheter after a heating step following the engaging step shown in FIG. 7(a);

FIG. 7(c) is a partial cross-sectional view diagrammatically illustrating a catheter after a shaping step following the heating step shown in FIG. 6(b);

FIG. 8(a) is a cross-sectional elevational partial view diagrammatically illustrating one embodiment of a double lumen catheter according to the present invention;

FIG. 8(a) is a sectional view taken along B-B in FIG. 8(a); and

FIG. 8(c) is a perspective view of the double lumen catheter of FIG. 8(a).

### DETAILED DESCRIPTION OF THE PREFERRED EMBODIMENTS OF THE INVENTION

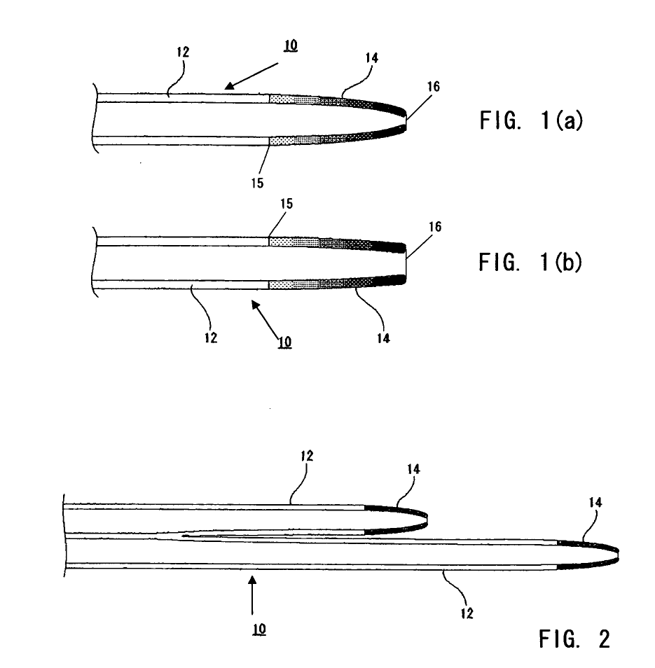

Referring now to the drawings, first to FIG. 1(a), designated generally as 10 is a catheter according to the present invention. The catheter 10 includes a tubular body 12 having a distal end 15 and a tubular tip portion 14 extending contiguously from the distal end 15 of the tubular body 12 substantially coaxially therewith and terminating in a forward facing aperture 16. The tubular tip portion 14 is formed of a water-absorbing, water-swelling material which absorbs water and swells when contacted with a water-containing liquid such as blood.

The catheter before contacting with water is rigid and, therefore, can be easily inserted into blood vessels. Upon insertion into and placement in blood vessel, the tip portion 14 of the catheter absorbs water and swells and, thus, softens. Therefore, the tip portion 14 of the catheter 10 no longer causes injury of blood vessel walls, even when the blood vessel moves by throb or even when the tip portion 14 is displaced for relocation. Further, upon swelling, the mouth of the tip portion 14 is radially enlarged as shown in FIG. 1(b), thereby increasing the rate of fluid flow therethrough. Additionally, the enlargement of the tip portion 14 can avoid clogging with a fibrin sleeve and thrombus in that region. The catheter of the present invention can be inserted into and placed in position in blood vessel by any conventional suitably method using an introducer sheath or by Seldinger method.

Any customarily employed resin which is substantially non-swellable in water may be suitably used to form the tubular body 12. Specific examples of the resin include non-hydrophilic or hydrophobic resins, such as hydrophobic polyurethane resins. Particularly preferred hydrophobic polyurethane resin comprises a recurring unit represented by the formula shown below and composed of dicyclohexylmethane diisocyanate as a hard segment and polyoxytetramethylene glycol and 1,4-butanediol as a soft segment:

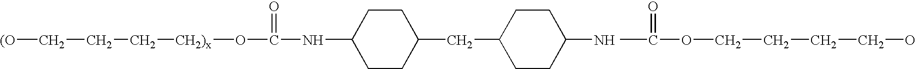

The water-absorbing, water-swelling material for forming the tip portion 14 is generally a resin comprising hard segments similar to those of the hydrophobic resin of the tubular body 12 and soft segments of a water-absorbing, water-swelling polyurethane. Particularly preferred water-absorbing, water-swelling resin comprises a recurring unit represented by the formula shown below and composed of dicyclohexylmethane diisocyanate as a hard segment and polyethylene glycol and 1,4-butanediol as a soft segment: 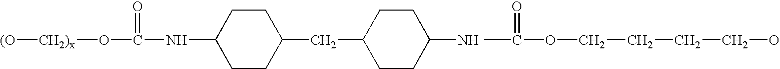
Any other water-absorbing, water-swelling resins, such as those disclosed in U.S. Pat. Nos. 4,454,309, 4,728,322 and 4,911,691, may be used for the purpose of the present invention.

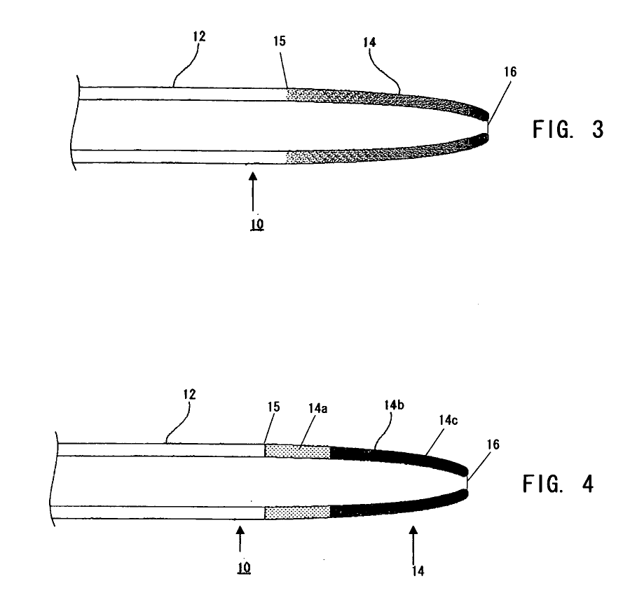

It is preferred that the water absorptivity or water absorbing efficiency of the tubular tip portion 14 increases in the direction from the distal end 15 of the tubular body 12 toward the open end (aperture) 16 of the tubular tip portion 14, as shown in FIG. 3. As a consequence of the gradual change of the water absorptivity, the softness of the tip portion 14 in a swollen state gradually increases toward the aperture 16. Therefore, a portion of the tip portion 14 adjacent to the aperture 16 can be made very soft while avoiding a reduction of mechanical strengths in the remainder portion (root portion and intermediate portion) of the tip portion 14 and bending of the tip portion in a root portion and intermediate portion thereof.

Such a tubular tip portion 14 having water absorptivity gradually increasing toward the aperture 16 may be formed by various methods.

In one preferred method, as shown in FIG. 4, a tubular tip portion 14 is obtained from two or more rings (three rings 14a, 14b and 14c in the illustrated embodiment) having water absorptivity increasing in the order of 14a, 14b and 14c. The water absorptivity may be controlled by, for example, controlling the proportion of the hydrophobic polyurethane relative to the water-absorbing, water-swelling polyurethane. The rings 14a-14c are connected in series by fuse-bonding or with an adhesive. If necessary, the connected rings 14a-14c are subjected to thermo-forming.

In an alternative method, a catheter is obtained by a method which comprises the steps of engaging an end portion of a tubular body made of a hydrophobic resin with an end portion of a tube made of a water-absorbing, water-swelling resin which is compatible with said hydrophobic resin, and heating and fusing the engaged end portions of the tubular body and the tube together, preferably while pressing them to each other, so that the tube is fuse-bonded to the tubular body at the engaged end portions. The resulting fuse-bonded engaged portion has a water absorptivity higher than that of the remainder portion of the tubular body but lower than that of remainder portion of the tube.

The engagement of an end portion of the tubular body with an end portion of the tube may be suitably carried out in the following manners.

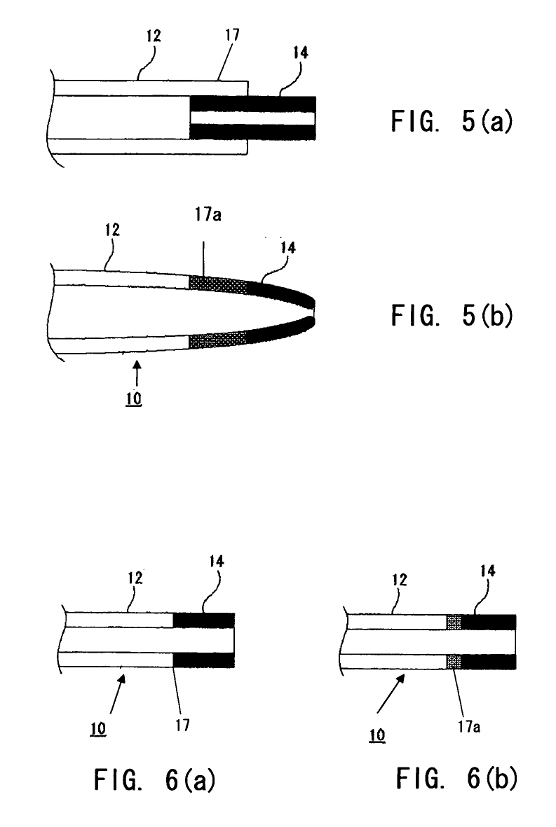

Thus, as shown in FIG. 5(a), an end portion of a tube 14 is fitted into a tubular body 12 to form an overlapped portion 17. Alternatively, an end portion of a tubular body 12 may be fitted into a tube 14. The overlapped portion 17 is then heated while being pressed to form a fuse-bonded engaged portion 17a, as shown in FIG. 5(b), having a water absorptivity higher than that of the remainder portion of the tubular body 12 but lower than that of remainder portion of the tube 14. If necessary, the resulting assembly is subjected to thermo-forming to obtain a smooth distant end.

As shown in FIG. 6(a), the engaging of an end portion of the tubular body 12 with an end portion of the tube 14 may be by abutting an end face of the tubular body 12 against and end face of the tube 14. Each of the end faces is normal to the axial direction of the tube. The abutted ends are heated to form a fuse-bonded engaged portion 17a, as shown in FIG. 6(b).

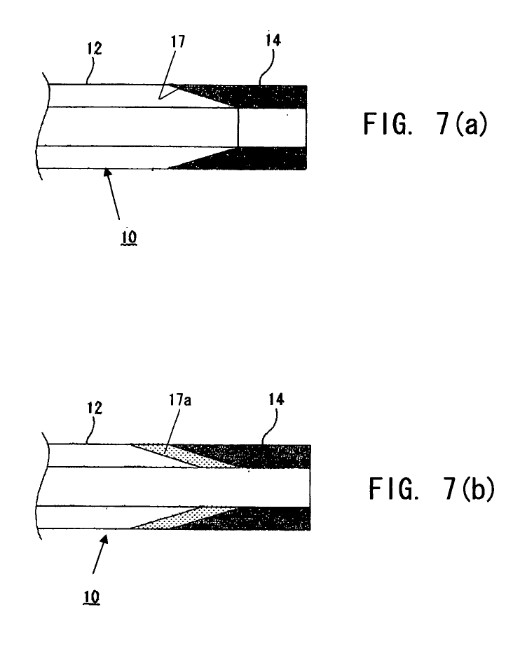

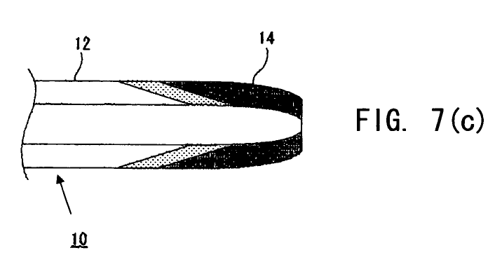

The abutting ends may be formed into slanted ends. Thus, as shown in FIG. 7(a), the end face of the tubular body 12 is outwardly inclined from the exterior surface to the interior surface thereof, while the end face of the tube 14 is inwardly inclined from the exterior surface to the interior surface thereof such that the inclined faces are face-to-face engageable with each other. The abutted ends are then heated to form a fuse-bonded engaged portion 17a, as shown in FIG. 7(b). If necessary, the resulting assembly is subjected to thermo-forming to obtain a smooth distant end as shown in FIG. 7(c).

The tip portion 14 of the catheter 10 according to the present invention may contain powder of an X-ray contrast medium or agent, such as barium sulfate, to provide radiopacity. The incorporation of the agent into the tip portion 14 may be performed by kneading the agent with a water-absorbing, water-swelling resin to obtain a kneaded resin composition, and using the resin composition for forming the tip portion 14. The tip portion 14 containing such an agent is suitably covered with a coating of a water permeable resin, preferably a water-absorbing, water-swelling resin similar to that used in the tip portion 14. Thus, because of the presence of the resin cover, the X-ray contrast medium is prevented from being released into blood. Because of the water-permeability of the resin cover, the tip portion 14 is able to absorb water and to swell.

The structure of the catheter of the present invention is not specifically limited. The tubular body 12 generally has an outer diameter in the range of about 1 to about 3 mm and the tip portion 14 generally has a length in the axial direction in the range of about 2 to about 10 mm.

The catheter may be constructed to have two tubular bodies 12, as illustrated in FIG. 2 in which the same reference numerals as those in FIG. 1(a) designate similar component parts. Each of the tubular bodies 12 is provided with a tip portion 14 formed of a water-absorbing, water-swelling material.

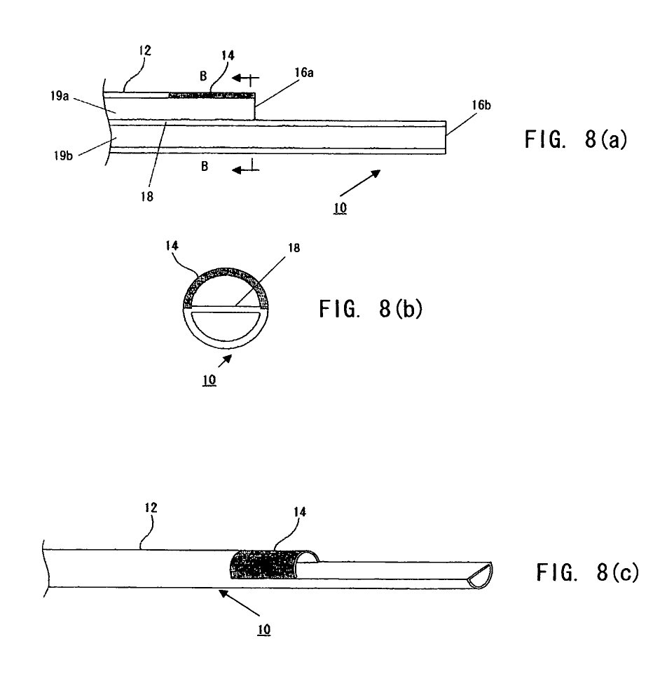

The catheter of the present invention may be constructed into a multi-lumen structure. FIGS. 8(a) through 8(c) depict an embodiment of a double lumen catheter which includes a tubular body 12, and a septum, generally a planar septum 18 disposed in the tubular body 12 to divide the interior of the tubular body into a first lumen 19a and a second lumen 19b. The first and second lumens 19a and 19b terminate in distally forward facing apertures 16a and 16b such that the aperture 16b of the second lumen 19b is spaced in the distally forward of the aperture 16a of the first lumen 19a. A tip portion 14 of the tubular body 12 which defines, together with the planar septum 18, a distal end part of the first lumen 19a including the aperture 16a is formed of a water-absorbing, water-swelling material.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description, and all the changes which come within the meaning and range of equivalency of the claims are therefore intended to be embraced therein.

1. A catheter comprising a tubular body having a distal end, and a tubular tip portion extending contiguously from said distal end substantially coaxially therewith and terminating in a forward facing aperture, said tubular tip portion being formed of a water-absorbing, water-swelling material.

2. A catheter as claimed in claim 1, wherein said tubular body is formed of a resin which does not substantially swell in water.

3. A catheter as claimed in claim 1, wherein the water absorptivity of said tubular tip portion increases in the direction from said distal end of said tubular body toward said aperture of said tubular tip portion.

4. A catheter as claimed in claim 3, wherein said tubular tip portion is obtained from two or more rings having different water absorptivity and connected in series such that the water absorptivity increases in the direction from said distal end of said tubular body toward said aperture of said tubular tip portion.

5. A catheter as claimed in claim 4, wherein said connection is by using an adhesive or by fuse-bonding.

6. A catheter as claimed in claim 4, wherein each of said rings are formed of a hydrophobic polyurethane and a water-absorbing water-swelling polyurethane and wherein the weight ratio of the hydrophobic polyurethane to the water-absorbing, water-swelling polyurethane of said rings varies so that said rings have different water absorptivities.

7. A catheter as claimed in claim 3, wherein said catheter is obtained by a method comprising the steps of engaging an end portion of a tubular body made of a hydrophobic resin with an end portion of a tube made of a water-absorbing, water-swelling resin which is compatible with said hydrophobic resin, and heating and fusing the engaged end portions of said tubular body and said tube together, so that said tube is fuse-bonded to said tubular body at said engaged end portions, and wherein said fuse-bonded engaged end portions have a water absorptivity higher than that of the remainder portion of said tubular body but lower than that of the remainder portion of said tube.

8. A catheter as claimed in claim 7, wherein said engaging of the end portion of said tubular body with the end portion of said tube is by abutting an end of said tubular body against an end of said tube.

9. A catheter as claimed in claim 8, wherein one of the abutting ends of said tubular body and said tube is inwardly inclined and wherein the other one of the abutting ends is outwardly inclined such that the abutting ends being face-to-face engaged with each other.

10. A catheter as claimed in claim 7, wherein said engaging of the end portion of said tubular body with the end portion of said tube is by fitting the end portion of said tubular body into said tube.

11. A catheter as claimed in claim 7, wherein said engaging of the end portion of said tubular body with the end portion of said tube is by fitting the end portion of said tube into said tubular body.

12. A catheter as claimed in claim 1, wherein said water-absorbing, water-swelling material further comprises a powder of an X-ray contrast medium, and wherein said tubular tip portion is covered with a water permeable resin coat.

13. A catheter as claimed in claim 12, wherein said water permeable resin is a water-absorbing, water-swelling resin.

14. A double lumen catheter, comprising
a tubular body; and
a septum disposed in said tubular body to divide the interior of said tubular body into a first lumen and a second lumen, said first and second lumens terminating in distally forward facing apertures such that the aperture of said second lumen is spaced in the distally forward of the aperture of said first lumen,
wherein a tip portion of said tubular body which defines, together with said septum, a distal end part of said first lumen including said aperture thereof is formed of a water-absorbing, water-swelling material.

15. A double lumen catheter as claimed in claim 14, wherein said water-absorbing, water-swelling material further comprises a powder of an X-ray contrast medium, and wherein said tip portion of said tubular body is covered with a water permeable resin coat.

16. A double lumen catheter as claimed in claim 15, wherein said water permeable resin is a water-absorbing, water-swelling resin.
